(19) United States
(12) Patent Application Publication
(10) Pub. No.: US 2020/0071286 A1
(43) Pub. Date: Mar. 5, 2020

(54) UTILIZATION OF NORMAL CARBON 4 (NC4) RECYCLE STREAM FOR SECONDARY AND TERTIARY PRODUCTS

(71) Applicant: SABIC Global Technologies B.V., Bergen op Zoom (DE)

(72) Inventors: Guillermo LEAL, Riyadh (SA); Mohammed Bismillah ANSARI, Riyadh (SA); Vijay BODAS, Riyadh (SA); Sultan AL OTAIBE, Riyadh (SA)

(21) Appl. No.: 16/467,178

(22) PCT Filed: Dec. 13, 2017
(86) PCT No.: PCT/IB2017/057907

§ 371 Date: Jun. 6, 2019

Related U.S. Application Data
(60) Provisional application No. 62/436,545, filed on Dec. 20, 2016.

(51) Int. Cl. C07D 307/60; C07D 307/33; C07D 307/08; C07C 29/17

(52) U.S. Cl. CPC C07D 307/60; C07D 307/33; C07C 31/207; C07C 29/17; C07D 307/08

(57) ABSTRACT

Certain embodiments are directed to an integrated process for the production of Maleic Anhydride (MAN), 1,4 Butanediol (BDO), Gamma-ButyroLactone (GBL), and PolyButylene Terephthalate (PBT) utilizing NC4 rich stream from a recycle stream after or before processing by a Total Hydrogenation Unit (THU).

### CROSS REFERENCE TO RELATED APPLICATIONS

This application claims the benefit of priority of U.S. Provisional Patent Application No. 62/436,545, filed Dec. 20, 2016, which is hereby incorporated by reference in its entirety.

### BACKGROUND

Light olefins such as ethylene, propylene, and butadiene are important basic raw materials in the petrochemical industry and are mainly produced by the furnace steam cracking process. Statistics shows that about 99% of ethylene, more than 50% of propylene and more than 90% of butadiene in the world are produced by this process. Cracking is a process whereby carbon-carbon bonds in saturated petroleum hydrocarbons are broken down or dehydrogenated under high temperature into olefins and other products. The object of cracking is to produce ethylene and propylene, with side products such as butene, butadiene, and other olefins, and pyrolysis gasoline, diesel, fuel oil, etc. There remains a need for processing and recycling product streams in a more efficient manner.

### SUMMARY

The actual recycle streams of the steam crackers has been not utilize fully and can be more fully utilized by providing for secondary and tertiary products from the normal 4 carbon (NC4) stream. Various elements of the invention described herein can be used to maximize infrastructure utilization and process efficiency by utilizing NC4 streams. In certain aspects the methods can utilize steam crackers as a source for NC4 streams. The utilization of these NC4 recycles will increase secondary and tertiary streams and provide for production of additional products. In certain aspects the NC4 stream can comprise 1-butene from 5 to 60 wt %, 2-trans-butene from 2 to 20 wt %, 2-cis-butene from 2 to 20 wt %, n-butane from 3 to 40 wt %, isobutene from 1 to 20 wt %, isobutene from 0.1 to 20 wt %, and 1,3-butadiene from 0.1 to 30 wt %.

Certain embodiments are directed to an integrated process for the production of Maleic Anhydride (MAN), 1,4 Butanediol (BDO), Gamma-ButyroLactone (GBL), and PolyButylene Terephthalate (PBT) utilizing NC4 rich stream from recycle C4's stream after or before processing by a Total Hydrogenation Unit (THU). The integrated process can comprise passing feedstock and product streams in a sequence through particular zones integrating the various processes and utilizing the feed and product streams more efficiently.

Certain aspects of the process provides crude MAN as feedstock for BDO, Tetrahydrofuran (THF), and/or GBL production. BDO and purified terephthalic acid (PTA) can be utilized as feedstocks for a PolyButylene Terephthalate (PBT) unit for production of PBT and THF. In certain aspects over 25 kilotons per annum (kta) of BDO, 60 kta of PBT, and 0.3 kta of THF can be produced using one or more process configuration described herein. In still a further aspect BDO can be further process to produced N-methylpyrrolidone (NMP), N-ethylpyrrolidone (NEP), and 2-pyrrolidone). GBL (gamma-butyrolactone), tetrahydrofuran (THF) and Polybutylene terephthalate (PBT).

Certain embodiments are directed to a process or methods of forming products from C4 hydrocarbons in an olefin producing plant; the method comprising: (a) receiving, from a hydrogenation unit, a hydrocarbon stream comprising primarily C4 hydrocarbons; (b) contacting, in a reactor unit, the hydrocarbon stream with a catalyst under reaction conditions to form maleic anhydride; and (c) converting, in one or more finished product units, at least some of the maleic anhydride to one or more of: 1,4 butanediol (BDO), gamma-butyrolactone (GBL), or tetrahydrofuran (THF), wherein the hydrogenation unit is in fluid communication with the reactor unit and the reactor unit is in fluid communication with the one or more finished product units.

Other embodiments are directed to methods of forming products from C4 hydrocarbons in an olefin producing plant that utilizes a steam cracker unit; the method comprising: (a) receiving, from a hydrogenation unit, a hydrocarbon stream comprising primarily C4 hydrocarbons, wherein the primary C4 hydrocarbon in the hydrocarbon stream is n-butane; (b) contacting, in a reactor unit, the hydrocarbon stream with a catalyst under reaction conditions to form maleic anhydride; (c) converting, in one or more finished product units, at least some of the maleic anhydride to one or more of: 1, 4 butanediol (BDO), gamma-butyrolactone (GBL), or (THF), wherein the hydrogenation unit is in fluid communication with the reactor unit and the reactor unit is in fluid communication with the one or more finished product units; and (d) reacting at least some of the BDO to form polybutylene terephthalate (PBT).

Certain embodiments are directed to methods of forming products from n-butane in an olefin producing plant that utilizes a steam cracker unit; the method comprising: (a) receiving, from a hydrogenation unit, a hydrocarbon stream comprising primarily n-butane, wherein no portion of the hydrocarbon stream is recycled to the steam cracker unit; (b) contacting, in a reactor unit, the hydrocarbon stream with a catalyst under reaction conditions to form maleic anhydride; (c) converting, in one or more finished product producing units, at least some of the maleic anhydride to one or more of: 1, 4 butanediol (BDO), gamma-butyrolactone (GBL), or tetrahydrofuran (THF), wherein the hydrogenation unit is in fluid communication with the reactor unit and the reactor unit is in fluid communication with the one or more finished product units; (d) reacting at least some of the BDO with purified terephthalic acid (PTA) to form polybutylene terephthalate (PBT); and (e) converting at least some of the BDO to one or more of: N-methylpyrrolidone (NMP), N-ethylpyrrolidone (NEP), or 2-pyrrolidone.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “primarily” as that term is used in the specification and/or claims, means greater than 50%, e.g., 50 wt. %, 50 mol. %, and/or 50 vol. %, etc., for example, from 50.01 to 100.00%, preferably 51% to 99%, and more preferably 60% to 90%.

In the context of the present invention, twenty embodiments are now described. Embodiment 1 is a method of forming products from C4 hydrocarbons in an olefin producing plant. The method includes the steps of receiving, from a hydrogenation unit, a hydrocarbon stream containing primarily C4 hydrocarbons; contacting, in a reactor unit, the hydrocarbon stream with a catalyst under reaction conditions to form maleic anhydride; and converting, in one or more finished product units, at least some of the maleic anhydride to one or more of: 1,4 butanediol (BDO), gamma-butyrolactone (GBL), or tetrahydrofuran (THF), wherein the hydrogenation unit is in fluid communication with the reactor unit and the reactor unit is in fluid communication with the one or more finished product units. Embodiment 2 is the method of embodiment 1, wherein the feed stream for the hydrogenation unit is a side product stream received from a butane separation unit. Embodiment 3 is the method of embodiment 2, wherein the feed stream for the butane separation unit is a side stream received from a methyl tertiary butyl ether (MTBE) production unit. Embodiment 4 is the method of embodiment 3, wherein a feed stream for the MTBE production unit is received from a selective hydrogenation unit (SHU). Embodiment 5 is the method of embodiment 4, wherein a feed stream for the SHU is received form a butadiene production unit. Embodiment 6 is the method of embodiment 5, wherein a feed stream for the butadiene production unit is received from a debutanizer unit coupled to steam cracking unit. Embodiment 7 is the method of embodiment 5, wherein a feed stream for the butadiene production unit is received from a steam cracking unit.

Embodiment 8 is a method of forming products from C4 hydrocarbons in an olefin producing plant that utilizes a steam cracker unit. This method includes the steps of receiving, from a hydrogenation unit, a hydrocarbon stream containing primarily C4 hydrocarbons, wherein the primary C4 hydrocarbon in the hydrocarbon stream is n-butane; contacting, in a reactor unit, the hydrocarbon stream with a catalyst under reaction conditions to form maleic anhydride; converting, in one or more finished product units, at least some of the maleic anhydride to one or more of: 1,4 butanediol (BDO), gamma-butyrolactone (GBL), or (THF), wherein the hydrogenation unit is in fluid communication with the reactor unit and the reactor unit is in fluid communication with the one or more finished product units; and reacting at least some of the BDO to form polybutylene terephthalate (PBT). Embodiment 9 is the method of embodiment 8, wherein the feed stream for the hydrogenation unit is a side product stream received from a butane separation unit. Embodiment 10 is the method of embodiment 9, wherein the feed stream for the butane separation unit is a side stream received from a methyl tertiary butyl ether (MTBE) production unit. Embodiment 11 is the method of embodiment 10, wherein a feed stream for the MTBE production unit is received from a selective hydrogenation unit (SHU). Embodiment 12 is the method of embodiment 11, wherein a feed stream for the SHU is received form a butadiene production unit. Embodiment 13 is the method of embodiment 12, wherein a feed stream for the butadiene production unit is received from a debutanizer unit coupled to steam cracking unit. Embodiment 14 is the method of embodiment 12, wherein a feed stream for the butadiene production unit is received from a steam cracking unit.

Embodiment 15 is a method of forming products from n-butane in an olefin producing plant that utilizes a steam cracker unit. The method of embodiment 15 includes the steps of receiving, from a hydrogenation unit, a hydrocarbon stream containing primarily n-butane, wherein no portion of the hydrocarbon stream is recycled to the steam cracker unit; contacting, in a reactor unit, the hydrocarbon stream with a catalyst under reaction conditions to form maleic anhydride; converting, in one or more finished product producing units, at least some of the maleic anhydride to one or more of: 1, 4 butanediol (BDO), gamma-butyrolactone (GBL), or (THF), wherein the hydrogenation unit is in fluid communication with the reactor unit and the reactor unit is in fluid communication with the one or more finished product units; reacting at least some of the BDO with purified terephthalic acid (PTA) to form polybutylene terephthalate (PBT); and converting at least some of the BDO to one or more of: N-methylpyrrolidone (NMP), N-ethylpyrrolidone (NEP), or 2-pyrrolidone. Embodiment 16 is the method of embodiment 15, wherein the feed stream for the hydrogenation unit is a side product stream received from a butane separation unit. Embodiment 17 is the method of embodiment 16, wherein the feed stream for the butane separation unit is a side stream received from a methyl tertiary butyl ether (MTBE) production unit. Embodiment 18 is the method of embodiment 17, wherein a feed stream for the MTBE production unit is received from a selective hydrogenation unit (SHU). Embodiment 19 is the method of embodiment 18, wherein a feed stream for the SHU is received form a butadiene production unit. Embodiment 20 is the method of embodiment 19, wherein a feed stream for the butadiene production unit is received from a debutanizer unit coupled to steam cracking unit, or directly from a steam cracking unit.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustrates a schematic of one embodiment of the invention diagramming integrated process that minimizes inefficiency in processing a steam cracking product stream. Abbreviation used include: H₂=hydrogen; MeOH=methanol; MTBE=methyl tertiary butyl ether; PTA=purified terephthalic acid; PBT=polybutylene terephthalate; MAN=maleic anhydride; BDO=1,4 butanediol; GBL=gamma-butyrolactone; THF=tetrahydrofuran; SHU=selective hydrogenation unit, and THU=total hydrogenation unit.

### DESCRIPTION

The full utilization of recycle streams has been always a major target for optimization. The elimination of recycles, in general, always improves the efficiency of the recipient unit opening the door for increasing actual capacity and better technical and operational performance. The processes, systems, and methods described herein utilize the NC4 recycle stream for secondary and tertiary production, which increases efficiency in the utilization of infrastructure. In certain aspects the utilization of NC4 to eliminate/minimize the recycle option in the steam crackers is targeted and used for production of secondary and tertiary products. In certain aspects these multiple products from NC4 increases production and expands products. Production can be increased by increasing available volumes to greater than 10 kilo-ton per hour (KTPH) of rich NC4 stream. The NC4 stream can be provided by a THU using the steam cracker recycle stream as feedstock for a maleic anhydride (MAN) unit, with an estimate of around 44,000 ton/year of MAN and with production of 60.14 tons/hour of high pressure steam (HPS) at 725 psig leading to 5.5 Ton/Hour fuel savings. This HPS production from MAN unit can reduce fuel consumption and utilization of two boilers.

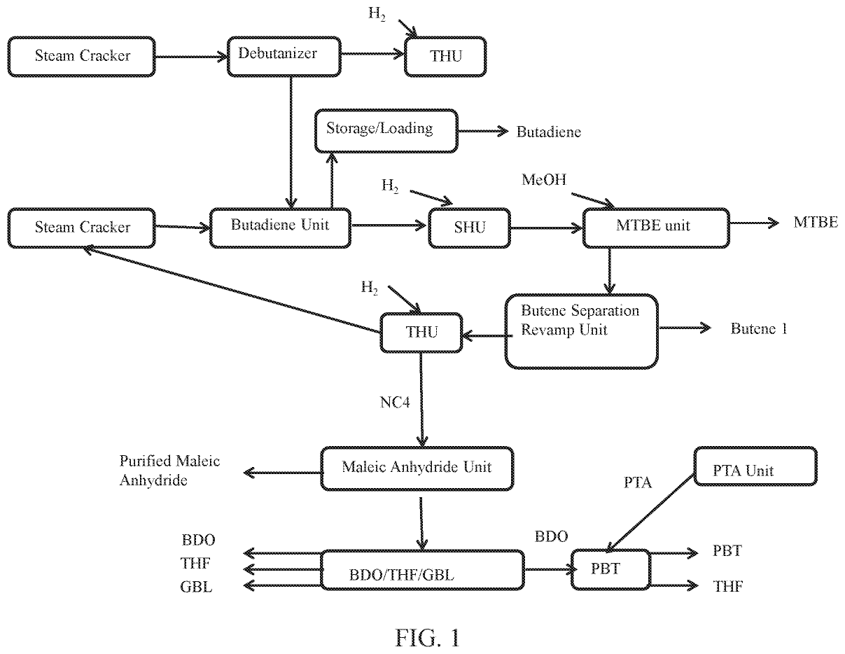

The NC4 stream can be augmented by including or integrating other appropriate systems, processes, and appropriate recycle or side-product streams. These other streams can be obtained from other units working in parallel or in series, as well as collecting recycle streams from other facilities. As shown in FIG. 1, an alternate or additive NC4 source can be derived from a steam cracker indirectly coupled to a MAN unit by selecting a number streams or side products from a series of processing units. The steam cracker is coupled to a debutanizer that is coupled to a butadiene unit. The butadiene unit is indirectly coupled to the MAN unit via a selective hydrogenation unit (SHU), MTBE conversion unit, a butane separation unit, and the THU upstream of the MAN unit. This additional volume could increase the proposed Maleic Anhydride plant production capacity up to 64,000 ton/year with 87.47 tons/hour of HPS at 725 psig, resulting in 8 ton/hour fuel savings. It is estimated from operational data that one ton of fuel gas from reboilers produces approximately 11-12 tons of HPS (100 bar).

In another embodiment a process, which can be integrated with other processes or systems, can be used where hydrogenated olefins from C4 hydrogenation unit is recycled and co-cracked in the steam cracker unit. In certain aspects a NC4 stream from the THU can be introduced into a MAN unit. The MAN unit can provide for a feedstock for: (i) an established BDO/THF/GBL unit, or (ii) partial or total BDO feed for a PBT unit that together with a PTA stream can be used as feedstocks for PBT production. Utilizing the design described herein integration between a steam cracker complex and purified terephthalic acid (PTA) is achieved. MAN (Maleic Anhydride Technology). Utilization of crude MAN form the MAN unit can be a source of feedstock for downstream process and systems.

An estimated 50% of world maleic anhydride output is used in the manufacture of unsaturated polyester resins (UPR). Chopped glass fibers are added to UPR to produce fiberglass reinforced plastics which are used in a wide range of applications such as pleasure boats, bathroom fixtures, automobiles, tanks and pipes.

GBL and BDO are widely used industrial chemicals serving as critical ingredients in many different products and applications. GBL offers excellent solvent qualities with low toxicity and diminished environment concerns. GBL is involved in the manufacture of products including hospital supplies, beverage filtration and purification aids. GBL is also used for applications including circuit board cleaning in the electronics and high technology industries; the production of herbicides; and as a processing aid in the production of vitamins and pharmaceuticals. While there are solvent applications for GBL, the majority of the GBL manufactured is used by industrial companies as an intermediate in the manufacturing process of other chemicals.

THF is a moderately polar aprotic solvent. It finds its application as a solvent for adhesives, lacquers, printing ink and unvulcanized rubber. It is used as a chemical intermediate in the preparation of polytetramethylene glycol; butyrolactone; succinic acid; adipic acid; 1.4 butanediol diacetate; and tetrahydrothiophene.

PTA is the main raw material for polyethylene terephthalate (PET) and polyester fibers, and is produced by oxidizing paraxylene. Some of the most common uses of PET include food, beverage, and consumer good packaging. Polyester fibers are used mainly in rugs, clothing, furniture and industrial applications, as well as other consumer products.

By using the process and design described herein each of these secondary or tertiary products can be produced in a more efficient and cost effective manner. Each unit will comprise the necessary devices, reactors, and apparatus for performing the process assigned to that particular unit as well as including all valves, piping, tubing, pumps, etc. needed to introduce feed streams and move products, side-products, or recycle streams to and from units in order to perform the processes described herein.

1. A method of forming products from C4 hydrocarbons in an olefin producing plant; the method comprising:
receiving, from a hydrogenation unit, a hydrocarbon stream comprising primarily C4 hydrocarbons;
contacting, in a reactor unit, the hydrocarbon stream with a catalyst under reaction conditions to form maleic anhydride; and
converting, in one or more finished product units, at least some of the maleic anhydride to one or more of: 1,4 butanediol (BDO), gamma-butyrolactone (GBL), or tetrahydrofuran (THF), wherein the hydrogenation unit is in fluid communication with the reactor unit and the reactor unit is in fluid communication with the one or more finished product units.

2. The method of claim 1, wherein the feed stream for the hydrogenation unit is a side product stream received from a butane separation unit.

3. The method of claim 2, wherein the feed stream for the butane separation unit is a side stream received from a methyl tertiary butyl ether (MTBE) production unit.

4. The method of claim 3, wherein a feed stream for the MTBE production unit is received from a selective hydrogenation unit (SHU).

5. The method of claim 4, wherein a feed stream for the SHU is received from a butadiene production unit.

6. The method of claim 5, wherein a feed stream for the butadiene production unit is received from a debutanizer unit coupled to steam cracking unit.

7. The method of claim 5, wherein a feed stream for the butadiene production unit is received from a steam cracking unit.

8. A method of forming products from C4 hydrocarbons in an olefin producing plant that utilizes a steam cracker unit; the method comprising:
receiving, from a hydrogenation unit, a hydrocarbon stream comprising primarily C4 hydrocarbons, wherein the primary C4 hydrocarbon in the hydrocarbon stream is n-butane;
contacting, in a reactor unit, the hydrocarbon stream with a catalyst under reaction conditions to form maleic anhydride;
converting, in one or more finished product units, at least some of the maleic anhydride to one or more of: 1,4 butanediol (BDO), gamma-butyrolactone (GBL), or (THF), wherein the hydrogenation unit is in fluid communication with the reactor unit and the reactor unit is in fluid communication with the one or more finished product units; and
reacting at least some of the BDO to form polybutylene terephthalate (PBT).

9. The method of claim 8, wherein the feed stream for the hydrogenation unit is a side product stream received from a butane separation unit.

10. The method of claim 9, wherein the feed stream for the butane separation unit is a side stream received from a methyl tertiary butyl ether (MTBE) production unit.

11. The method of claim 10, wherein a feed stream for the MTBE production unit is received from a selective hydrogenation unit (SHU).

12. The method of claim 11, wherein a feed stream for the SHU is received from a butadiene production unit.

13. The method of claim 12, wherein a feed stream for the butadiene production unit is received from a debutanizer unit coupled to steam cracking unit.

14. The method of claim 12, wherein a feed stream for the butadiene production unit is received from a steam cracking unit.

15. A method of forming products from n-butane in an olefin producing plant that utilizes a steam cracker unit; the method comprising:
receiving, from a hydrogenation unit, a hydrocarbon stream comprising primarily n-butane, wherein no portion of the hydrocarbon stream is recycled to the steam cracker unit;
contacting, in a reactor unit, the hydrocarbon stream with a catalyst under reaction conditions to form maleic anhydride;
converting, in one or more finished product producing units, at least some of the maleic anhydride to one or more of: 1, 4 butanediol (BDO), gamma-butyrolactone (GBL), or (THF), wherein the hydrogenation unit is in fluid communication with the reactor unit and the reactor unit is in fluid communication with the one or more finished product units;
reacting at least some of the BDO with purified terephthalic acid (PTA) to form polybutylene terephthalate (PBT); and
converting at least some of the BDO to one or more of: N-methylpyrrolidone (NMP), M-ethylpyrrolidone (NEP), or 2-pyrrolidone.

16. The method of claim 15, wherein the feed stream for the hydrogenation unit is a side product stream received from a butane separation unit.

17. The method of claim 16, wherein the feed stream for the butane separation unit is a side stream received from a methyl tertiary butyl ether (MTBE) production unit.

18. The method of claim 17, wherein a feed stream for the MTBE production unit is received from a selective hydrogenation unit (SHU).

19. The method of claim 18, wherein a feed stream for the SHU is received from a butadiene production unit.

20. The method of claim 19, wherein a feed stream for the butadiene production unit is received from a debutanizer unit coupled to steam cracking unit, or directly from a steam cracking unit.
